(19) United States
(12) Patent Application Publication
(10) Pub. No.: US 2025/0098942 A1
(43) Pub. Date: Mar. 27, 2025

(54) SYSTEMS, DEVICES, AND METHODS FOR CALIBRATING A MEDICAL CAMERA

(71) Applicant: Stryker Corporation, Portage, MI (US)

(72) Inventors: Ruth GODBEY, Sunnyvale, CA (US); Cole Kincaid HUNTER, Helena, MT (US); Brian FOUTS, Morgan Hill, CA (US); Suvarna Shyamal MAJI, Navi Mumbai (IN)

(73) Assignee: Stryker Corporation, Portage, MI (US)

(21) Appl. No.: 18/894,322

(22) Filed: Sep. 24, 2024

Related U.S. Application Data
(60) Provisional application No. 63/585,191, filed on Sep. 25, 2023.

(51) Int. Cl. A61B 1/00

(52) U.S. Cl. CPC A61B 1/00154; A61B 1/00045; A61B 1/00057

(57) ABSTRACT

A method includes displaying visual guidance for guiding a user through an endoscopic camera calibration process. The visual guidance guides the user to direct an endoscopic camera at a plurality of different regions of a field of calibration markers. Images captured by the endoscopic camera while the user directs the endoscopic camera at the calibration markers are received. Suitability of images for use in calibration for the endoscopic camera is determined. In accordance with determining that at least one image is suitable for use in calibration, the visual guidance is updated to indicate to the user that the respective region of the plurality of different regions of the field of calibration markers has been successfully imaged. Calibration factors for calibrating the endoscopic camera are determined based on images determined to be suitable for use in calibration for the endoscopic camera.

### CROSS-REFERENCE TO RELATED APPLICATIONS

This application claims the benefit of U.S. Provisional Patent Application No. 63/585,191, filed Sep. 25, 2023, the entire contents of which is hereby incorporated by reference herein.

### FIELD

This disclosure generally relates to medical imaging and, more particularly, to calibrating a medical camera.

### BACKGROUND

Endoscopic imaging involves the use of a high-definition camera coupled to an endoscope inserted into a patient to provide a surgeon with a clear view within the body. The images collected by the camera, which can be video or individual snapshot images, can be transmitted to a display device that renders the images collected onto a display so that the surgeon can visualize the internal area of the body that is being viewed by the camera. The surgeon may depend on the visualization provided by the camera to perform a procedure in the internal area of the patient using one or more tools that are specifically configured to aid the surgeon as they perform the procedure. The surgeon can view the images being displayed to them during a surgery to manipulate and navigate the tool within the internal area of the body.

Endoscopic images can also be used by the surgeon to measure distances or determine the position of an object within the internal area of the body. For instance, if the scale of the images shown on a display is known, as well as depth information, then the surgeon can use the endoscopic images to measure distances of the internal area of the body in either two dimensions, three dimensions, or both. In instances where sufficient information about endoscopic images exists to measure distances or determine the position of an object in the image, one or more tools used within the internal area of the body during an endoscopic procedure can be used to measure distances and/or determine the position of a feature in the internal area of the patient. A pointer tool is an example of a tool that can be used by a surgeon during an endoscopic procedure. A pointer tool can include a tip that the surgeon can use to palpate the anatomy of the patient and act as the “fingers” of the surgeon during an endoscopic surgery. The surgeon can use the tip of the pointer tool to measure distance in the anatomy or otherwise determine the two- or three-dimensional location of a feature of a patient's anatomy.

Endoscopic cameras are susceptible to image distortion, which can negatively impact the accuracy and effectiveness of measurements generated from images captured during a surgical procedure. Calibration of the endoscopic camera can be used to correct for such distortion and can be particularly important in surgeries that require high levels of precision. Conventional calibration techniques generally involve using the endoscopic camera to capture an image of an object having known dimensions. Differences in the dimensions of the object in the image compared to its known dimensions can be used to remove distortion from images captured by the endoscopic camera.

### SUMMARY

According to an aspect, systems, devices, and methods guide a user through a calibration process for capturing a plurality of quality calibration images for use in calibrating a medical camera, such as an endoscopic camera. A graphical user interface may guide the user through the use of a calibration fixture to capture images of a field of calibration markers that have known dimensions. The calibration fixture may be configured to aid the user in positioning the endoscope suitably for capturing quality calibration images. The user may be guided to image different regions of the field of calibration markers so that calibration images having multiple viewing angles are generated. Images captured by the endoscopic camera during the calibration process may be analyzed to determine their suitability for use in calibrating the camera. The user may be provided with indications regarding the progress of the calibration process, such as indications that suitable images have been successfully captured or notifications of problems that may be occurring in capturing suitable images. Guiding a user through a process for capturing a plurality of calibration images and checking the calibration images for suitability can result in the collection of multiple high-quality calibration images that can provide high-quality and/or robust image distortion correction.

Images captured during the calibration process can be used to generate calibration factors for use in correcting distortion in images captured during a surgical procedure. Additionally or alternatively, images captured during the calibration process can be used to check whether predetermined calibration factors (e.g., calibration factors generated in a previous calibration process or default calibration factors) are sufficient for use in correcting for distortion. If they are not, then the user may be guided through a full calibration process.

According to an aspect, a method for guiding a user through an endoscopic camera calibration process includes, at a computing system: displaying visual guidance for guiding the user through the endoscopic camera calibration process, the visual guidance configured to guide the user to direct an endoscopic camera at a plurality of different regions of a field of calibration markers; receiving images captured by the endoscopic camera while the user directs the endoscopic camera at the plurality of different regions of the field of calibration markers; determining suitability of at least some of the images for use in calibration for the endoscopic camera; in accordance with determining that at least one image of a respective region of the plurality of different regions of the field of calibration markers is suitable for use in calibration for the endoscopic camera, updating the visual guidance to indicate to the user that the respective region of the plurality of different regions of the field of calibration markers has been successfully imaged; and determining calibration factors for calibrating the endoscopic camera based on images determined to be suitable for use in calibration for the endoscopic camera.

The visual guidance may include region indicators that correspond to the plurality of different regions of the field of calibration markers, and updating the visual guidance to indicate to the user that the respective region of the plurality of different regions of the field of calibration markers has been successfully imaged may include modifying a region indicator corresponding to the respective region of the plurality of different regions of the field of calibration markers. Updating the visual guidance to indicate to the user that the respective region of the plurality of different regions of the field of calibration markers has been successfully imaged may include displaying a graphical indicator within a field of view that comprises the respective region of the plurality of different regions of the field of calibration markers.

The method may include, in accordance with determining that a given image of a given region of the plurality of different regions of the field of calibration markers is not suitable for use in calibration for the endoscopic camera, displaying at least one notification for guiding the user to adjust use of the endoscopic camera. The at least one notification may include a notification that the endoscopic camera is too close to the field of calibration markers, that the endoscopic camera is out of focus, and/or that a rotation angle of the endoscopic camera is out of range.

Determining suitability of at least some of the images for use in calibration for the endoscopic camera may include determining a rotation angle of an endoscope of the endoscopic camera relative to a camera head of the endoscopic camera and comparing the rotation angle to a predetermined range of suitable rotation angles. Determining suitability of at least some of the images for use in calibration for the endoscopic camera may include detecting calibration markers and comparing a number of detected calibration markers to a threshold number of calibration markers associated with suitability of an image for calibration.

The method may include determining that each region of the plurality of different regions of the field of calibration markers has been successfully imaged, wherein the calibration factors are determined in response to determining that each region of the plurality of different regions of the field of calibration markers has been successfully imaged. The visual guidance may be configured to guide the user to direct the endoscopic camera at the plurality of different regions of the field of calibration markers from a plurality of different endoscopic camera positions. The visual guidance may indicate to the user an order in which to direct the endoscopic camera at the plurality of different regions of the field of calibration markers.

According to an aspect, a method for endoscopic camera calibration includes, at a computing system: receiving at least one image of at least a portion of a field of calibration markers captured by an endoscopic camera; undistorting the at least one image based on predetermined calibration factors for the endoscopic camera; determining relative positions of at least some of the calibration markers in the at least one undistorted image; comparing the relative positions of the at least some of the calibration markers to at least one predetermined range; in accordance with the relative positions of the at least some of the calibration markers not being within the at least one predetermined range, entering a recalibration mode for determining at least one calibration factor; and in accordance with the relative positions of the at least some of the calibration markers being within the at least one predetermined range, entering an imaging mode in which the predetermined calibration factors are used to modify images captured by the endoscopic camera. The relative positions of the at least some of the calibration markers may include relative positions of corresponding portions of the at least some of the calibration markers.

Determining the relative positions of at least some of the calibration markers in the at least one undistorted image may include: determining a location of a portion of a first calibration marker in the at least one undistorted image and a location of a corresponding portion of a second calibration marker in the at least one undistorted image; and determining a distance from the location of the corresponding portion of the second calibration marker to a line extending from the location of the portion of the first calibration marker, wherein the relative positions of at least some of the calibration markers in the at least one undistorted image comprises the distance from the location of the corresponding portion of the second calibration marker to the line extending from the location of the portion of the first calibration marker. The line may be a horizontal line and the distance may be a vertical distance or the line may be a vertical line and the distance is a horizontal distance. The method may include determining a scaling factor based on a size of at least one of the first calibration marker and the second calibration marker in the at least one undistorted image and predetermined size information associated with the at least one of the first calibration marker and the second calibration marker, wherein the distance from the location of the corresponding portion of the second calibration marker to the line extending from the location of the portion of the first calibration marker is determined based on the scaling factor.

The at least one image of at least a portion of the field of calibration markers may include a plurality of images of different regions of the field of calibration markers, wherein relative positions of calibration markers are determined and compared for each of the different regions of the field of calibration markers. The predetermined calibration factors may include calibration factors determined during a previous calibration process. The predetermined calibration factors may be associated with an endoscopic camera model or identifier.

A method for endoscopic camera calibration may include, at a computing system: receiving at least one image of at least a portion of a field of calibration markers captured by an endoscopic camera; determining a position of a rotational orientation indicator in the at least one image that corresponds to a rotational orientation of an endoscope of the endoscopic camera relative to a camera head of the endoscopic camera; comparing the determined position of the rotational orientation indicator to at least one range of positions associated with suitability of the at least one image for use in determining calibration factors for the endoscopic camera; in accordance with the determined position of the rotational orientation indicator not being within the at least one range of positions, displaying a graphical indicator that indicates that the endoscope is not properly oriented; and in accordance with the determined position of the rotational orientation indicator being within the at least one range of positions displaying a graphical indicator that indicates that the endoscope is properly oriented.

The method may include determining the calibration factors for the endoscopic camera based only on images having rotational orientation indicators positioned within the at least one range of positions. The at least one range of positions may include two ranges of positions, and the calibration factors may be determined based on a first set of images having the rotational orientation indicators positioned within a first range of positions and a second set of images having the rotational orientation indicators positioned within a second range of positions.

The rotational orientation indicator may include a notch located at a periphery of a field of view portion of the at least one image. Determining the position of the rotational orientation indicator in the at least one image comprises detecting the rotational orientation indicator, wherein detecting the rotational orientation indicator comprises: determining a circle that corresponds to the periphery of the field of view portion of the at least one image; determining a contour that encompasses the notch; and determining a point on the contour that is furthest from a centroid of the contour.

The method may include displaying visual guidance to a user indicating proper orientation of the endoscope. The visual guidance may include a graphical target overlayed on the at least one image, wherein the graphical target is positioned where the rotational orientation indicator should be positioned for the proper orientation of the endoscope.

According to an aspect, a system includes one or more processors and memory storing one or more programs for execution by the one or more processors, the one or more programs including instructions that, when executed by the one or more processors, cause the system to perform any of the above methods.

According to an aspect, a non-transitory computer readable storage medium stores instructions for execution by one or more processors of a computing system, which when executed by the one or more processors, cause the system to perform any of the above methods.

It will be appreciated that any of the variations, aspects, features, and options described in view of the systems apply equally to the methods and vice versa. It will also be clear that any one or more of the above variations, aspects, features, and options can be combined.

### BRIEF DESCRIPTION OF THE FIGURES

The invention will now be described, by way of example only, with reference to the accompanying drawings, in which:

FIG. 1 illustrates an exemplary endoscopy system;

FIG. 2 illustrates an example of the use of a calibration fixture for use in a camera calibration process;

FIG. 3 illustrates an example of calibration features that can be used for the calibration fixture of FIG. 2;

FIG. 4 illustrates an example of a calibration fixture incorporating the calibration marker field of FIG. 3;

FIG. 5 is a flow diagram of an exemplary method 500 for guiding a user through an endoscopic camera calibration process;

FIGS. 6A-6D illustrate exemplary graphical user interfaces for guiding a user through an endoscopic calibration process;

FIG. 7 is a flow diagram of an exemplary method for guiding a user to capture a field of fiducial markers with different endoscope rotational orientations;

FIG. 8 is a flow diagram of an exemplary method for determining whether predetermined calibration factors are sufficient for use in correcting distortion;

FIG. 9 illustrates an example of the determination of the alignment of corners of calibration markers;

FIG. 10 illustrates an exemplary coordinate system for a pointer of an exemplary pointer tool;

FIG. 11 illustrates an exemplary coordinate system for a pointer of an exemplary pointer tool;

FIGS. 12A and 12B illustrate an exemplary ArUco marker that can be disposed on a pointer tool; and

FIG. 13 is a block diagram of an exemplary computing system.

### DETAILED DESCRIPTION

Reference will now be made in detail to implementations and examples of various aspects and variations of systems and methods described herein. Although several exemplary variations of the systems and methods are described herein, other variations of the systems and methods may include aspects of the systems and methods described herein combined in any suitable manner having combinations of all or some of the aspects described.

Described herein, according to various aspects, are systems, devices, and methods for calibrating endoscopic cameras. A camera calibration process includes capturing a plurality of calibration images that are used for generating calibration factors for correcting distortion in images. A graphical user interface may guide a user through a series of steps for capturing quality calibration images using a calibration fixture that includes a field of calibration markers. The graphical user interface may provide instructions and/or other guidance for using the calibration fixture to capture images of the field of calibration markers from multiple different angles. As the user is stepping through the calibration process, images are assessed to determine if they are suitable for use in calibration. The process can help ensure that quality calibration images are captured, enabling the generation of reliable and robust calibration factors.

A calibration fixture may be configured for positioning an endoscope within a range of distances from a field of calibration markers that corresponds to the typical working range of an endoscope during an endoscopic surgery. The calibration fixture may include a plurality of ports for positioning the endoscope in different positions relative to the field of calibration markers. The field of calibration markers may have a plurality of different regions of calibration markers for a user to image.

A graphical user interface may include one or more graphical indicators that indicate to a user how to capture images using the calibration fixture. The graphical user interface may guide the user through a series of steps for capturing images of each of the regions of calibration markers. Where the calibration fixture has multiple ports, the graphical user interface may indicate to the user when to move the endoscope to a different port.

As the user is capturing images (e.g., video) of the field of calibration markers, the images may be analyzed to determine whether they are suitable for use in calibrating the endoscopic camera. The calibration markers in the images may be detected, and aspects of the detection of the calibration markers may be used to determine whether a given image is suitable for use in calibration. For example, an image generated when the endoscope is too close to the field of calibration markers may be determined unsuitable due to it having too few calibration markers. Conversely, an image generated when the endoscope is too far from the field of calibration markers may be determined unsuitable due to it having too many calibration markers. Optionally, a user may be given a notification or other indication associated with the reason that an image is determined to be unsuitable. For example, a notification may be provided in the graphical user interface that the endoscope is too close or too far.

The user may be guided through the calibration process at any suitable time, such as during set-up of an operating room in preparation for a surgical procedure, after a power cycle of the camera, after an endoscope has been switched out, or at any other time. In some instances, predetermined calibration factors may still be suitable and a full calibration process may be skipped, thus saving the user (e.g., surgeon, nurse, or other medical personnel) time. A user may be guided through a pre-calibration process in which the user captures images of the field of calibration markers, the images are corrected using the predetermined calibration factors, and the quality of the corrected images is assessed to determine if the calibration factors are still satisfactory. If they are not, the user may be guided through the full calibration process. If the predetermined calibration factors are satisfactory, the full calibration process is skipped.

The systems, devices, and method described herein can generate better-quality calibration factors than conventional systems and methods. In conventional systems and methods, the quality of the images used in calibrating is generally not controlled, resulting in poor-quality images. For example, the calibration images in conventional systems can have very few known points or can be out of focus. They can have robustness limitations due to not viewing the calibration object from multiple angles. The images can be out of the actual targeted working distance. In some cases, only a single image is used. Any one of these quality issues can lead to poor accuracy and/or poor robustness of calibration factors. Additionally, conventional systems and methods do not check whether the full calibration process can be skipped in favor of predetermined calibration factors. The systems, device, and methods described herein improve upon these issues by guiding a user through a calibration process that captures multiple, high-quality calibration images of calibration markers from different perspectives, leading to higher-quality and/or more-robust calibration factors.

Camera calibration, according to the principles described herein, may also be used for other camera types, including open-field cameras, microscope cameras, and any other surgical and non-surgical camera types. Although the examples described herein often include a calibration fixture, the methods and systems are not limited to use with a calibration fixture. Rather, calibration markers may be provided in any location within an operating room (or other medical room) that is viewable by a camera, such as on a display in the operating room, on a wall in the operating room, on a panel of an imaging system component in the operating room, on a wall or other surface of a cart in the operating room, etc.

In the following description of the various examples, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units, but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating,” or the like refer to the action and processes of a computer system, or similar electronic computing device, that manipulates and transforms data represented as physical (electronic) quantities within the computer system memories or registers or other such information storage, transmission, or display devices.

The present disclosure in some examples also relates to a device for performing the operations herein. This device may be specially constructed for the required purposes, or it may comprise a general-purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application-specific integrated circuits (ASICs), or any type of media suitable for storing electronic instructions, and each connected to a computer system bus. Furthermore, the computing systems referred to in the specification may include a single processor or may be architectures employing multiple-processor designs, such as for performing different functions or for increased computing capability. Suitable processors include central processing units (CPUs), graphical processing units (GPUs), field programmable gate arrays (FPGAs), and ASICs.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present disclosure as described herein.

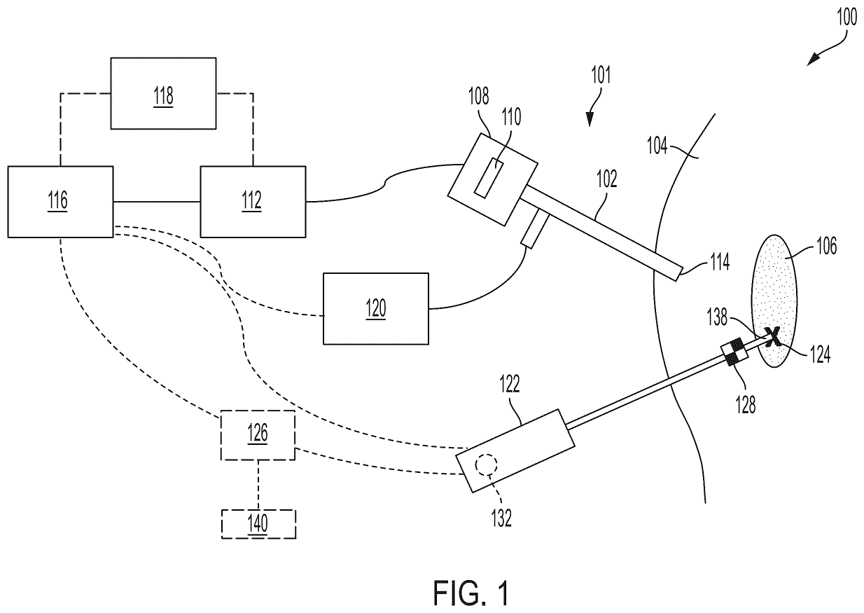

FIG. 1 illustrates an exemplary endoscopy system 100. System 100 includes an endoscopic camera 101 that can include a camera head 108 mounted to an endoscope 102. As is well known in the art, the endoscope 102 can be configured for insertion into a surgical cavity 104 for imaging tissue 106 within the surgical cavity 104 during a medical procedure. Light generated by a light source 120 may be directed through the endoscope 102 to the surgical cavity 104. Light reflected by and/or emitted from the tissue 106 (such as fluorescence light emitted from fluorescing targets that are excited by fluorescence excitation illumination light provided by the light source 120) is received at the distal end 114 of the endoscope 102. The light is propagated by the endoscope 102, such as via one or more optical components (for example, one or more lenses, prisms, light pipes, or other optical components), to the camera head 108, where it is directed onto one or more imaging sensors 110. One or more filters (not shown) may be included in the endoscope 102 in a coupler (not shown) connecting the endoscope 102 to the camera head 108, and/or in the camera head 108 for filtering a portion of the light received from the tissue 106 (such as fluorescence excitation light used for fluorescence imaging).

The one or more imaging sensors 110 generate pixel data that can be transmitted to a camera control unit 112 that is communicatively connected to the camera head 108. The camera control unit 112 can generate endoscopic images from the pixel data that shows the tissue being viewed by the endoscopic camera 101. (As used herein, “image” encompasses single-snapshot images and a video frame. As such, “images” encompasses multiple snapshot images and multiple video frames.) The endoscopic images can be transmitted to an image processing system 116 for further image processing, storage, display, and/or routing to an external device (not shown). The endoscopic images can be transmitted to one or more displays 118, from the camera control unit 112 and/or the image processing system 116, for visualization by medical personnel, such as by a user for visualizing the surgical cavity 104 during a surgical procedure on a patient. The camera control unit 112 and/or the image processing system 116 may be configured to send control signals to the light source 120 and/or the camera head 108 to control one or more aspects of the imaging, such as a timing sequence of light provided by the light source 120 (e.g., a sequence of white light and fluorescence excitation light), an amount of light provided by the light source 120, and/or a gain of the one or more imaging sensors 110.

The images generated by the endoscopy system 100 can be used to create two-dimensional and/or three-dimensional maps of the tissue 106 of the patient. For instance, in one or more examples, the images can be represented using an (x,y) coordinate system, in which each location or point of the tissue 106 can correspond to a specific (x,y) coordinate. Even as the endoscopic camera 101 is repositioned throughout a surgery, the images created by the endoscopic camera 101 can be stitched together to create an overall two-dimensional mapping of the tissue 106, such that no two points of the tissue 106 viewed by the endoscopic camera 101 will have the same (x,y) coordinate.

A two-dimensional model created from the endoscopic video feed during a surgical procedure can be transformed into a three-dimensional model by adding depth information to the two-dimensional model. Depth information pertaining to endoscopic image or endoscopic video feed can be obtained by using hardware-based methods such as employing the use of stereo cameras, time-of-flight sensors, etc. Additionally or alternatively, the depth information can be acquired algorithmically, for instance by using a structure from motion process in conjunction with a camera to acquire depth information. Additionally or alternatively, the depth information can be acquired using external data acquired on the patient such as magnetic resonance images (MRIs), etc. Similar to two-dimensional mappings, the above techniques can be employed to create a three-dimensional map of the internal anatomy of the patient, such that every point visualized by an endoscopic camera can have a unique (x,y,z) coordinate.

The two- and/or three-dimensional mappings discussed above can be used to generate two- or three-dimensional measurements within the internal portion of the patient. For instance, the distance between two (x,y,z) points within the patient's internal anatomy can be measured in real-time using the three-dimensional mappings acquired using the systems and processes described above. In order to take a measurement, a user may need to accurately identify the start point and end point of such a measurement, and/or the contours of the measurement to be taken. In one or more examples, a user can utilize a pointer tool 122 to point to the specific points in the internal anatomy of a patient to use in a two- or three-dimensional measurement that is being taken using images taken from an endoscopic imaging device. In one or more examples, the pointer tool 122 can include a pointer that has a tip 138 located at an end of the pointer tool 122 that can be captured in imaging data generated by the camera head 108 and used by the user to mark or point to a specific point of interest 124 in the imaging data of the patient's internal anatomy. One challenge associated with using a pointer tool to mark points in a patient's anatomy is identifying the precise location of the tip 138 in the endoscopic image.

To use the pointer as a “marking” device, an image processing system, such as image processing system 116, must determine where the tip of the pointer being used to mark is located. The task of finding the tip of the pointer tool can be even more complicated when the tip is obscured by a patient's anatomy (for instance, by being buried in the patient's tissue) or otherwise not completely visible in the endoscopic image due to other occlusions or obfuscations. To this end, the pointer tool 122 can be specifically configured to allow for easy and robust identification of the tip 138 of a pointer tool by an image processing system, such as image processing system 116, for the purposes of marking a portion of a patient's anatomy or any other context in which the precise two- and/or three-dimensional location of the tip 138 may be required. The pointer tool 122 can include features that enable an image processing system to acquire the location of the tip 138, regardless of the orientation the pointer tool 122 is in and regardless of whether the tip 138 is visible in the image or not. For example, the pointer tool 122 can include one or more fiducial markers 128 that can be captured in imaging data and used by an image processing system, such as image processing system 116, to not only identify the pointer tool 122, but also to identify its orientation and identify the precise two- or three-dimensional location of the tip of the tool, which the image processing system can use to take two- or three-dimensional measurements.

In some examples, the pointer tool 122 can include one or more buttons 132 or other user interface that a user can use to instruct the image processing system 116 to determine the position of the location of interest 124 based on the position of the tip 138 of the pointer tool 122. For example, the user can position the pointer tool 122 at or near the location of interest 124 and press the button 132 on the pointer tool 122 to indicate that the image processing system 116 should determine the position of the location of interest 124. The pointer tool 122 can be directly connected to the image processing system 116 or can be connected to a tool controller 126 configured to receive input from the pointer tool 122. The tool controller 126 can receive a signal from the pointer tool 122 responsive to a button press. The tool controller 126 can send a notification to the image processing system 116 indicative of the user's instruction to determine the location of interest 124. The image processing system 116 can then analyze one or more endoscopic images to determine the three-dimensional position of the location of interest 124. The user can reposition the pointer tool 122 and provide another button press to control the system 100 to determine a new location of interest based on the repositioned position of the pointer tool 122. This can be repeated any number of times by the user. In some examples, the pointer tool 122 may include a memory storing identifying information for the pointer tool 122 that the pointer tool 122 may provide to the image processing system 116 and/or the tool controller 126 so that the image processing system 116 and/or the tool controller 126 can determine how to interpret communications from the pointer tool 122.

In some examples, the pointer tool 122 does not include any user input features. Instead, the pointer tool 122 may include a shaft extending from a simple handpiece or simply a shaft grasped at one end by a user. In such examples, user input instructing the image processing system 116 to determine the three-dimensional position of the location of interest 124 can be provided via any other user interface of system 100, including, for example, a voice control system, a remote control, another tool, or a foot switch. For example, the tool controller 126 may include or be connected to a user interface 140, such as a foot switch, to which a user may provide an input to instruct the image processing system 116 to determine the three-dimensional position of the location of interest 124. Optionally, the tool controller 126 and user interface 140 can be used to communicate with tools other than the pointer tool 122, such as a cutting tool, and the tool controller 126 can change how it responds to inputs to the user interface 140 based on which tool is being used. The image processing system 116 may detect the presence of the pointer tool 122 in imaging data, such as by detecting the fiducial marker 128, and may inform the tool controller 126 that the pointer tool 122 is being used. The tool controller 126 may then respond to inputs to the user interface 140 based on configuration data associated with the pointer tool 122 (instead of, for example, configuration data associated with a cutter). Optionally, the configuration data may be customizable based on user preferences so that, for example, mappings of user interface 140 inputs to tool controller 126 outputs can be different for different users. Although FIG. 1 shows a pointer tool 122, it is to be understood that the principles described herein are applicable to any surgical tool, including cutting tools, drill guides, and/or any other surgical tool that can be placed in the surgical cavity within the field of view of an endoscopic camera.

Images generated by an endoscopic camera, such as endoscopic camera 101 of FIG. 1, often include some amount of distortion caused by physical attributes of the endoscopic camera. In order for an imaging processing system to generate accurate measurements, as discussed above, such distortion should be removed. The image processing system can use calibration factors to correct for distortion. At least some of the physical attributes that affect the distortion may change over time. For example, the coupling of a different endoscope to a camera head can result in changes to the light pathway to the image sensor that distorts image data in a different way. The systems, devices, and methods described herein can include a calibration process used to update calibration factors to account for changes in an endoscopic camera that can affect distortion. The calibration process could be conducted periodically, such as after a power cycle, at the beginning of a surgical imaging session, after removal and replacement of an endoscope, or at any other suitable time.

The calibration process generally includes capturing, with the endoscopic camera, images of one or more calibration features of a calibration fixture. The calibration features have known attributes (e.g., shapes, dimensions, relative positions, etc.) and can be or include, for example, one or more fiducial markers (also referred to herein as calibration markers). Attributes of the calibration features in the captured images are compared to the known attributes of those features, and differences between those attributes that are caused by distortion can be used to generate and/or update calibration factors for the endoscopic camera that can be used to correct for the distortion.

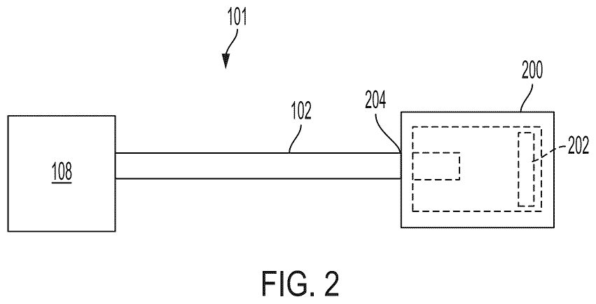

FIG. 2 illustrates an example of a calibration fixture 200 for use in a calibration process. The calibration fixture 200 includes one or more calibration features 202 for imaging by the endoscopic camera 101. The calibration fixture 200 can be configured to help a user (e.g., a surgeon, nurse, or other medical personnel) position the endoscopic camera 101 in a suitable position relative to the calibration features 202 so that the images of the calibration features 202 are suitable for generating calibration factors. For example, the calibration fixture 200 can include one or more ports 204 for receiving the endoscope 102, with the one or more ports 204 being positioned so that the endoscope 102 is in a suitable position (or range of positions) relative to the calibration features 202. In general, during a calibration process, a user may insert the endoscope 102 into a port 204 of the calibration fixture 200, the endoscopic camera 101 may capture one or more images of the calibration features 202, and the image processing system 116 may generate one or more calibration factors based on the one or more images. The calibration factors may then be used by the image processing system to generate images with corrected distortion.

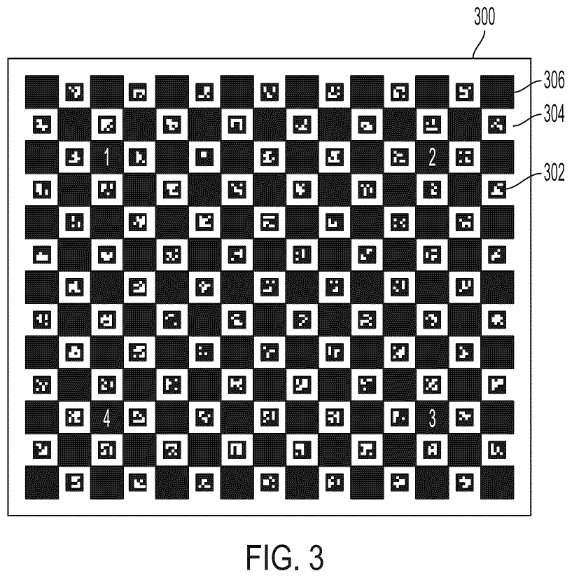

FIG. 3 illustrates an example of calibration features that can be used for the calibration features 202 in the calibration fixture 200 of FIG. 2. In the example of FIG. 3, the calibration features are a field 300 of calibration markers 302. The calibration markers 302 are positioned within light blocks 304 that are arranged in a checkerboard pattern with dark blocks 306. Attributes of the calibration markers 302, such as their size, spacing, and relative positions within the field 300, are predetermined and stored in a memory of, or accessible by, image processing system 116. Image processing system 116 can detect the calibration markers 302, determine attributes of the calibration markers 302 in images captured by the endoscopic camera 101, and use differences between the predetermined attributes of the calibration markers 302 and the attributes of the calibration markers 302 in the images to generate calibration factors.

In the illustrated example, the calibration marker field 300 includes four different regions of calibration markers, each having a region indicator (numbered 1 through 4 in the illustrated example). The endoscopic camera 101 may be directed at each of these four different regions in order to obtain images of markers from multiple different perspectives, which can help improve the generation of calibration factors. The numbering of the regions can help guide a user in directing the endoscopic camera 101 at the different regions (e.g., in numerical order). In other examples, the field 300 may include any number of region indicators suitable for obtaining images of calibration markers 302 from different perspectives. Though numerals are depicted in the illustrated example, the region indicator(s) can be any type of indicator, including symbols (e.g., circles, squares, etc.) and/or any other visual feature configured to prompt the user to point the camera at that region.

In the illustrated example, the calibration markers 302 are ArUco markers. ArUco markers can encode an identity of the ArUco marker that may uniquely identify the ArUco marker relative to other ArUco markers. The image processing system 116 can determine the identity of a given ArUco marker in an image based on its encoded identity and obtain, from a memory, attributes associated with that ArUco marker for use in generating the calibration factors. The ArUco marker may additionally comprise encoded error detection and correction information such that any discrepancies and/or errors in detecting the bit pattern of the ArUco marker may be minimized to determine the correct information associated with a given ArUco marker. Other examples of fiducial markers that may be used are bar codes, Quick Response (QR) codes, AprilTags, and/or any other fiducial markers having attributes suitable for generating calibration factors. In some examples, the calibration markers 302 may be printed in a dark color (e.g. black) on a light background. Alternatively, the calibration markers 302 may be printed in a color other than black, such as red, green or blue and the image processing system 116 may analyze one of the color channels of an image (the red, green, or blue channel of an image) to detect the calibration markers 302.

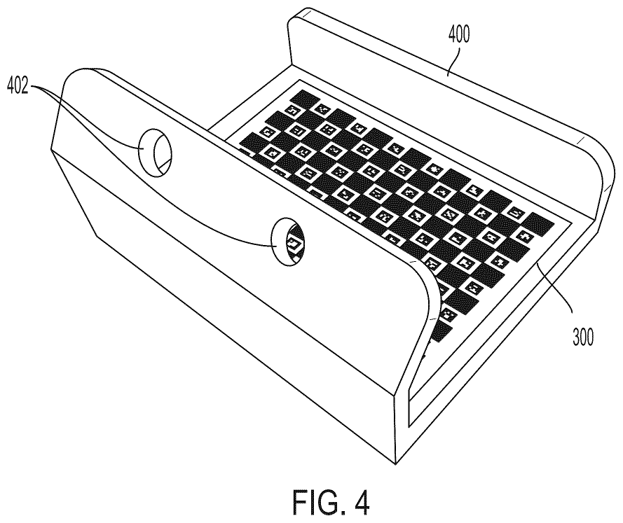

FIG. 4 illustrates an example of a calibration fixture 400 incorporating the calibration marker field 300 of FIG. 3. Calibration fixture 400 is configured to ensure that images of the calibration marker field 300 are captured at roughly a target working distance corresponding to a typical range of working distances experienced during surgery. The calibration fixture 400 includes one or more ports 402 for positioning the endoscope 102. Multiple ports 402 may be included as a means for guiding a user to capture images of the calibration marker field 300 using multiple different orientations of the endoscopic camera 101, which can help ensure quality of the calibration factors. The ports 402 are located and oriented so as to help a user position the endoscope 102 (e.g., the distal end 114 thereof) at a suitable distance and orientation relative to the calibration marker field 300. The ports 402 may be oriented at an angle to the calibration marker field 300 so that an angled distal end 114 of the endoscope 102 is oriented at an acute angle with respect to the calibration marker field 300 but not too shallow of an angle (e.g., less than 30°) to prevent detection of the respective calibration markers. Calibration fixture 400 may be a disposable component or a reusable component.

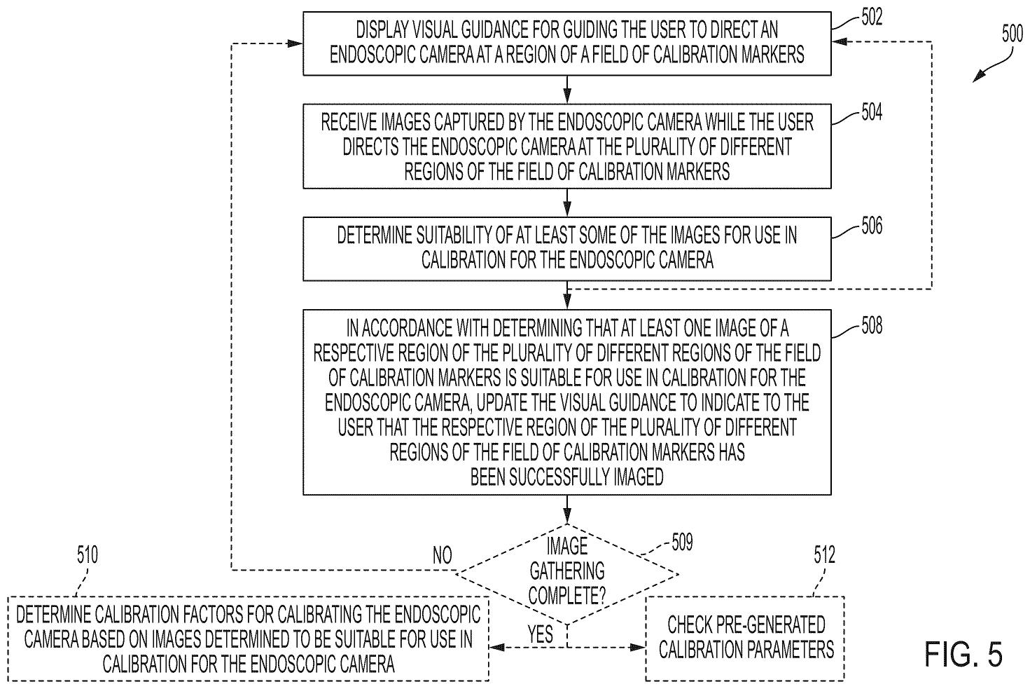

FIG. 5 is a flow diagram of an exemplary method 500 for guiding a user through an endoscopic camera calibration process. Method 500 may be performed by, for example, endoscopy system 100 of FIG. 1 for guiding a user, such as a surgeon, nurse, or other medical personnel, through a calibration process for calibrating endoscopic camera 101. One or more steps of method 500 may be performed, for example, by image processing system 116. Method 500 may be performed prior to using the endoscopic camera 101 for imaging a patient. For example, method 500 may be performed during preparation of an operating room for a surgical procedure or may be performed just prior to inserting the endoscopic camera 101 into a patient. Method 500 may be repeated as necessary. For example, method 500 may be repeated upon a user switching out the endoscope 102 of the endoscopic camera 101.

At step 502, visual guidance for guiding a user through an endoscopic camera calibration process is displayed on one or more displays, such as display 118 of FIG. 1. The visual guidance may be a graphical user interface that includes a live video feed captured by the endoscopic camera 101. The graphical user interface may include one or more textual and/or graphical indicators that indicate to the user that a calibration process is being executed or should be executed. The graphical user interface may, for example, instruct the user to initiate a calibration mode of the endoscopy system 100 and, in a response to a user selection for the calibration mode, may then guide the user through the calibration process, including by guiding the user to direct the endoscopic camera 101 at a field of calibration markers, such as the calibration marker field 300 of FIG. 3.

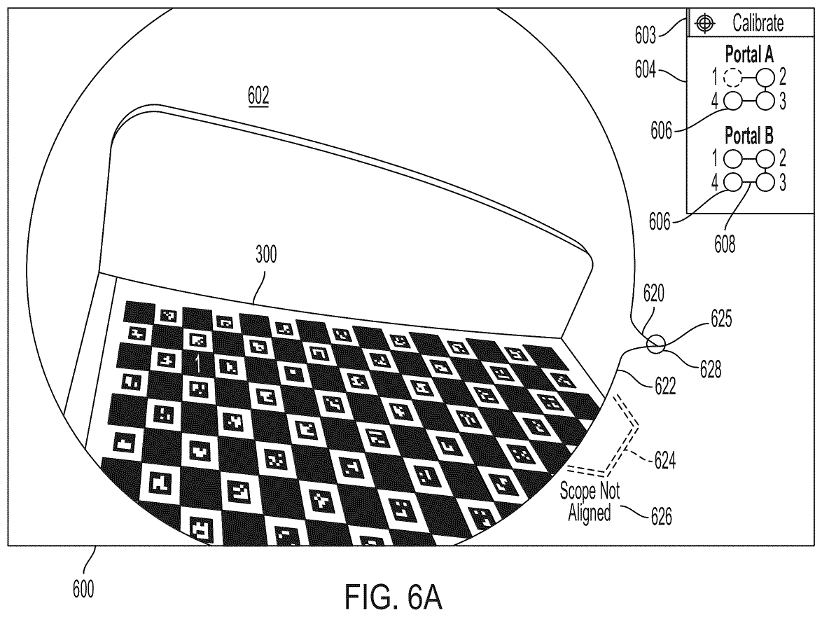

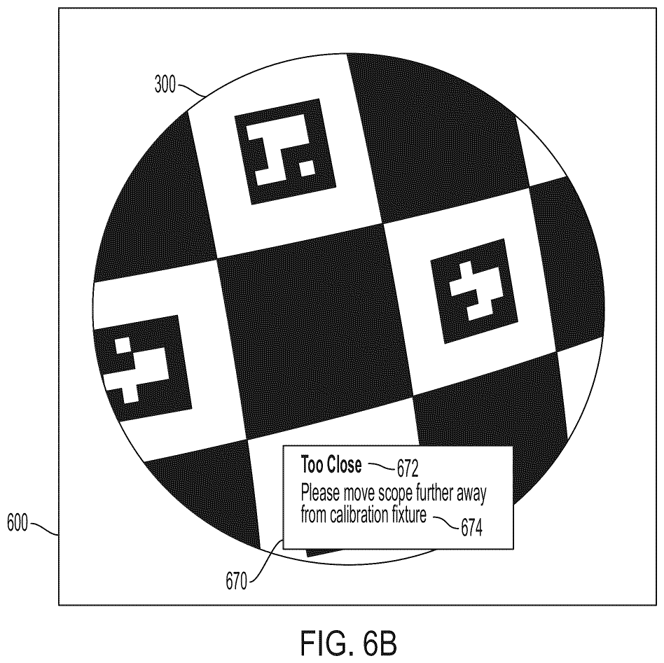

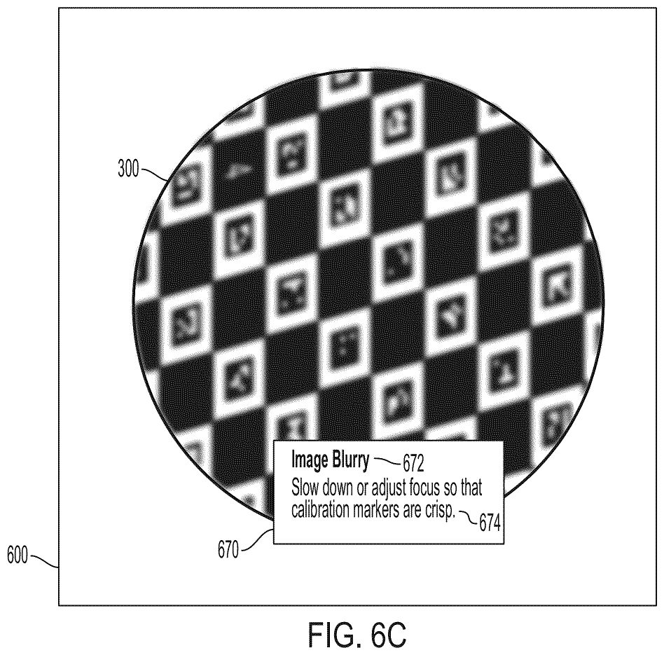

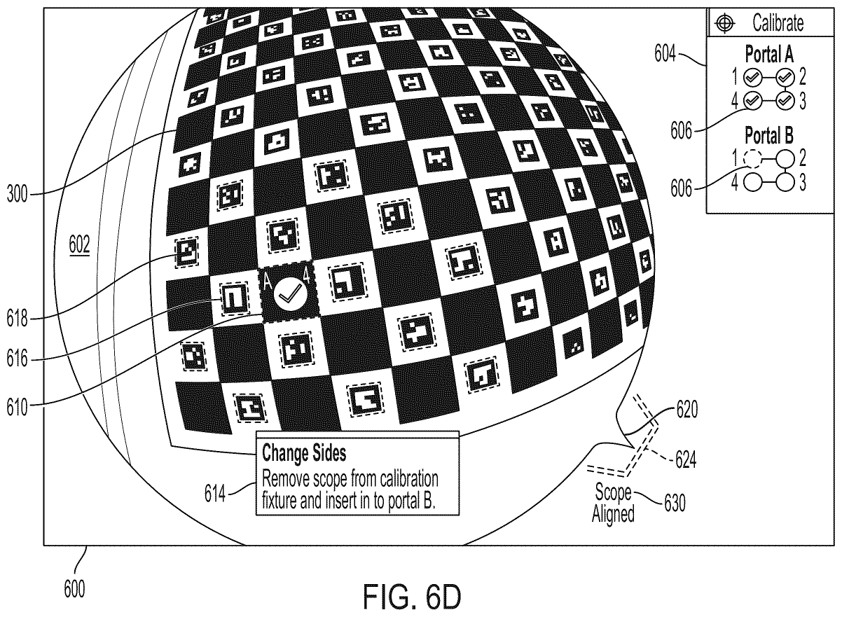

FIG. 6A illustrates an exemplary graphical user interface 600 that may be displayed according to step 502. The graphical user interface 600 includes a frame 602 of an endoscopic video being generated by the endoscopic camera 101. A first guidance portion 603 indicates to the user that the endoscopy system 100 is in a calibration mode. A second guidance portion 604 indicates to the user how to progress through the calibration process. The second guidance portion 604 may include textual and/or graphical indicators that are associated with a particular calibration fixture that the user is to use during the calibration process. In the illustrated example, the second guidance portion 604 specifies “Portal A” and “Portal B,” which may correspond to the two ports 402 of the calibration fixture 400 of FIG. 4. The circles 606—numbered 1 through 4—are examples of indicators of the four regions of the calibration marker field 300 of FIG. 3 and may indicate to the user that the user should direct the endoscopic camera 101 to those four regions, one at a time. Thus, the circles in combination with the “Portal A” and “Portal B” textual indicators can indicate to the user that the user should image the four regions of the calibration marker field 300 from each of the two ports 402 of the calibration fixture 400. The lines 608 between the circles 606 may indicate to a user the sequence in which the four regions of the calibration marker field 300 should be imaged. The visual appearance of a given circle 606 may change as the user successfully images the corresponding region of the calibration marker field 300.

At step 504, one or more images captured by the endoscopic camera 101 while the user directs the endoscopic camera 101 at the field of calibration markers are received at an image processing system, such as image processing system 116 of FIG. 1, or other computing system. For example, with reference to calibration fixture 400 of FIG. 4, the user may insert the endoscope 102 into one of the ports 402 while the endoscopic camera 101 is capturing video, and the one or more images can be frames of the video that capture the calibration marker field 300. The user may be directing the endoscope 102 at a particular region of the field of calibration markers, such as at any one of regions 1-4 of the calibration marker field 300, and the one or more images may include at least some of the fiducial markers in the region. Each image captured by the endoscopic camera 101 may be received by the image processing system in step 504. Alternatively, the image processing system may receive fewer than all the images captured by the endoscopic camera 101. For example, the image processing system may extract some, but not all, frames from a video stream captured by the endoscopic camera 101. The frames may be extracted at some regular interval (e.g., every other frame or every third frame) or can be extracted based on the processing of a previous frame (e.g., once analysis of a previous frame has completed, the image processing system may extract a new frame from the video stream). Extracting fewer than all of the frames may reduce processing time and/or computing resource demand.

At step 506, the image processing system may determine the suitability of at least some of the images received at step 504 for use in calibration for the endoscopic camera. The image processing system may analyze one or more images received at step 504 to detect the calibration markers in the image. The calibration markers can be detected according to any suitable image processing technique, such as by using a suitable edge-detection algorithm and searching for edges that correspond to features of the calibration markers and/or using a machine learning model trained to identify one or more calibration markers in endoscopic images. Detection of calibration markers may include extraction of information encoded by the calibration marker. For example, the calibration marker may be an ArUco marker having an encoding that uniquely identifies the ArUco marker relative to other ArUco markers, enabling the image processing system to identify the ArUco marker that it has detected.

The ability to detect the calibration markers and/or aspects of the detection of the calibration markers may factor into whether a given image is suitable for use in calibration or not. For example, an image may be determined to be unsuitable if no calibration markers can be detected. This could occur, for example, if an image is too blurry, such as due to excessive movement of the endoscopic camera, and/or if the endoscopic camera is out of focus. As another example, an image may be determined to be unsuitable if one or more calibration markers are detected but the number detected is insufficient. An insufficient number of calibration markers detected (e.g., below some predetermined threshold number of calibration markers associated with suitability of an image for use in calibration) may indicate that the endoscope 102 is too close to the field of calibration markers and/or is directed at least partially away from the field of calibration markers. Optionally, each of the calibration markers of a given region must be detected in a given image for the image to be determined suitable for use in calibration. Conversely, the detection of too many calibration markers (e.g., above a predetermined threshold) may indicate that the endoscope 102 is too far from the field of calibration markers.

The suitability of an image may be determined based on a given stage of a calibration process. For example, an image may be determined to be unsuitable if the image does not capture the region of the field of calibration markers that is needed at the given stage of the calibration process. As noted above, the calibration process may include capturing images of different regions of the field of calibration markers. An image that captures a different region than the one that is needed at a given stage may be determined to be unsuitable. The image processing system may determine whether the correct region has been captured in an image by identifying the particular calibration markers in the image. For example, the detection of a calibration marker in an image may provide the image processing system with the identity of the calibration marker, and if the calibration marker is not in the correct region, the image may be determined to be unsuitable.

In response to the image processing system determining that one or more images is unsuitable for use in calibration, the image processing system may provide feedback to the user that guides the user in obtaining a better image. For example, an alert may be displayed that includes an indication as to why a given image was determined to be unsuitable and/or what action or actions to take to obtain a better image. FIGS. 6B and 6C illustrate examples of alerts 670 that may be displayed in the graphical user interface 600 in response to the image processing system determining that the endoscope is too close to the calibration marker field 300 (FIG. 6B) and that the image is blurry (FIG. 6C). One or more of the alerts 670 may include an indication 672 of the reason the image is unsuitable and/or an indication 674 of an action a user can take to obtain a better image. If one or more images are determined to be unsuitable in step 506, method 500 may return to step 502 or step 504 for the user to capture more images.

Each image received at step 504 may be analyzed at step 506. Alternatively, some subset of the images received at step 504 may be analyzed at step 506. The frames that are analyzed at step 506 may be selected based on some regular interval (e.g., every other frame or every third frame) or may be selected based on the processing of a previous frame (e.g., once analysis of a previous frame has completed, the image processing system may analyze a new frame from the video stream).

At step 508, if at least one image has been determined to be suitable for use in calibration in step 506, the visual guidance is updated to indicate to the user successful completion of the step of the calibration process associated with at least one image. For example, where the calibration process includes steps for capturing images of different regions of a field of fiducial markers, the visual guidance can be updated to indicate to the user that a respective region of the field of calibration markers has been successfully imaged.

An example of an updated visual guidance that indicates that a respective region of the field of calibration markers has been successfully imaged is illustrated in FIG. 6D. In the illustrated example, the graphical user interface 600 has been updated to indicate that the four different regions of the calibration marker field 300 have been successfully captured in one or more images by an endoscope positioned in Portal A of the calibration fixture (e.g., calibration fixture 400). This is indicated by the modifications to the four circles 606 of Portal A to include checkmarks in second guidance portion 604. The graphical user interface 600 also includes a graphical indicator 610 overlaid on region 4 of the calibration marker field 300 of the frame 602 displayed in the graphical user interface 600. The graphical indicator 610 includes a checkmark indicating successful imaging and an “A” and “4” indicating region 4 from Portal A. The graphical user interface 600 may also include a notification 614 guiding the user to move the endoscope to “Portal B” of the calibration fixture. The graphical user interface 600 may also include outlines 616 positioned around each of the calibration markers 618 detected in the frame 602, which may indicate to the user that the image processing system has successfully detected those calibration markers.

Optionally, the capture of a single suitable image for a given region of the field of calibration markers may be sufficient for the calibration process, and, thus, the visual guidance may be updated according to step 508 once a single image has been determined to be suitable for use in calibration in step 506. Alternatively, multiple suitable images may be needed for each region of the field of calibration markers, and step 508 will occur once a predetermined number of images for a given region have been determined suitable.

Steps 504 through 508 may be performed repeatedly to guide the user to capture images of each of a plurality of regions of the field of calibration markers. The visual guidance may be updated throughout the process to guide the user through the capture of images of each of the regions. For example, once a threshold number of images of a given region of the field of calibration markers has been determined to be suitable for that region, method 500 may return to step 502, and the graphical user interface may be updated to indicate that the user should move to the next region. The method may then proceed through repeats of steps 504 through 508 for determining suitability of images for the next region until, for example, a determination is made that image gathering is complete at step 509 (e.g., a determination that a predetermined number of suitable images for all regions and all calibration fixture ports have been obtained).

Method 500 may include step 510 in which the images determined to be suitable in step 506 are used for determining calibration factors for calibrating the endoscopic camera. This may occur automatically, such as in response to determining that each region of the plurality of different regions of the field of calibration markers has been successfully imaged, or based on some user instruction, such as a user response to a prompt notifying the user that all suitable calibration images have been successfully captured. In determining the calibration factors, attributes of the calibration markers in the images can be compared to the known attributes of those calibration markers. Optionally, the graphical user interface can indicate to the user that the calibration process has successfully completed. Once the calibration factors have been successfully determined at step 510, the image processing system may exit a calibration mode and may enter an imaging mode. This may be automatic or may be based on a user selection from a menu of modes. Optionally, a notification may be provided in the graphical user interface indicating that the calibration has been successfully completed. If the determination of calibration factors in step 510 fails, the graphical user interface may notify the user of the failure and may guide the user into repeating the calibration process.

Determining the calibration factors according to step 510 may include generating an initial camera matrix and distortion coefficients based on the images determined to be suitable in step 506. Reprojection errors are then computed for each image, and the reprojection errors are compared to a threshold. Images with reprojection errors higher than the threshold are discarded. Next, calibration marker corner interpolation is performed using the initial camera matrix and distortion coefficients. If a sufficient number of corners are interpolated, then calibration marker-based calibration is performed using the initial camera matrix and distortion coefficients. From this, a final reprojection error is computed. If the final reprojection error is greater than an acceptable threshold, then the calibration has failed, and the user may be prompted to redo the calibration process. If the final reprojection error meets the acceptable threshold, the camera matrix and distortion coefficients are set as the calibration factors for use in undistorting images during the imaging mode.

As explained above, calibration factors may be generated based on images of a field of calibration taken from different perspectives. The generation of the calibration factors can be improved by guiding the user to image the field of calibration markers with different endoscope rotational orientations (i.e., angles about a longitudinal axis of the endoscope) since a user may adjust the orientation of the endoscope during a surgical procedure. An image generated by an endoscopic camera may include an indicator of the rotational orientation of the endoscope about the imaging axis relative to the endoscopic camera head. This indicator is useful for helping a user understand the relationship between how the user is positioning the endoscope and what the user is seeing in the images. The position of the indicator in an image can be used to determine the orientation of the endoscope relative to the camera head and to guide the user in changing the orientation of the endoscope for capturing the field of calibration makers with different endoscope orientations.

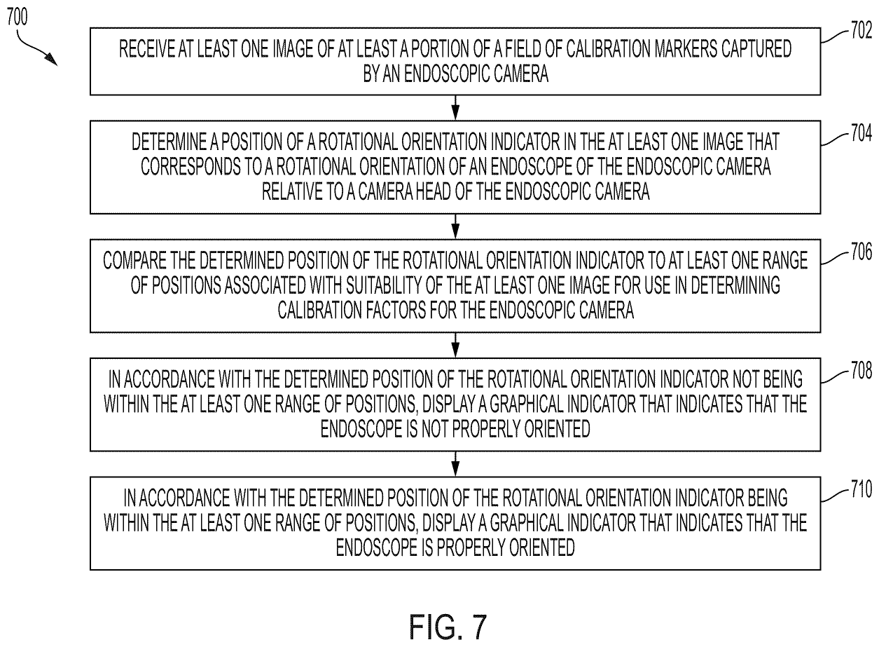

FIG. 7 is a flow diagram of an exemplary method 700 for guiding a user to capture a field of fiducial markers with different endoscope rotational orientations. Method 700 can be performed along with one or more steps of method 500 of FIG. 5. At step 702, at least one image of at least a portion of a field of calibration markers captured by an endoscopic camera may be received at an image processing system, such as image processing system 116 of FIG. 1. Step 702 is analogous to step 502 of method 500 and may be the same when methods 500 and 700 are performed together.

At step 704, a position of a rotational orientation indicator in the at least one image received at step 702 is determined. FIG. 6A illustrates an example of an endoscopic image having an endoscope rotational orientation indicator 620 (also commonly referred to as a carrot or a notch). As explained above, the location of the indicator 620 (i.e., its position around the circular perimeter of the field of view of the endoscopic image) corresponds to a rotational orientation of the endoscope 102 of the endoscopic camera 101 relative to a camera head 108 of the endoscopic camera 101. The image can be analyzed by the image processing system to detect the indicator 620 and determine its position.

To detect the position of the indicator 620, the image processing system may apply a suitable circle-detection algorithm that detects the circular perimeter 622 of the field of view of the image. For example, a machine learning model, such as a circle-detection segmentation model, may be used to generate an estimate of the circular perimeter 622. Next, a mask associated with the circular perimeter 622 is dilated to generate a contour that encompasses the indicator 620. A centroid of the circular perimeter 622 can be determined, and the location of the portion of the contour that is furthest from the center is the tip 625 of the indicator 620. The field of view of the image may then be divided into angular regions (e.g., from 0 to 360 degrees) centered at the center of the circular perimeter 622 of the field of view, and the angular region that includes the location of the indicator 620 may provide the position of the indicator 620 (i.e., the rotation angle of the endoscope relative to the camera head).

The position of the indicator 620 may be difficult to determine if, for example, the indicator 620 happens to overlap with a calibration marker that has a dark color (e.g., black) due to the similarity in the dark color of the calibration marker and the black of the area outside of the field of view of the image. To improve the detection of the indicator 620 in such a scenario, the calibration marker may be a color other than black, such as red, green, or blue. This increases the contrast between the calibration marker and the indicator 620, improving the detectability of the indicator 620. The image processing system may be configured to detect the calibration marker using one or more color channels of an image that correspond to the color of the calibration marker (e.g., one of the red, green, and blue color channels, depending on the color of the calibration markers).

At step 706, the determined position of the indicator 620 is compared to at least one predetermined range of positions (e.g., rotation angles) associated with suitability of the at least one image for use in determining calibration factors for the endoscopic camera. The predetermined range can include, for example, angular positions of the endoscope that are most commonly used during surgical procedures. Examples of such ranges include 208° to 220° or 323° to 335° (where 0° is aligned with the horizontal direction in the illustrated image). The predetermined range used for assessing the position of the indicator 620 may depend on the stage of the calibration process. For example, a first portion of the calibration process may include capturing images of the field of calibration markers with the endoscope in a first range of angular positions (e.g., 208° to 220°), a second portion of the calibration process may include capturing images of the field of calibration markers with the endoscope in a second range of angular positions (e.g., 323° to 335°), and the suitability of an image may be determined based on the range for the stage for which the image is being evaluated. For example, with reference to calibration fixture 400 of FIG. 4, the first range of angular positions may be used for images captured while the endoscope is positioned in one of the ports 402 and the second range of angular positions may be used for images captured while the endoscope is positioned in the other of the ports 402.

At step 708, in accordance with the determined position of the rotational orientation indicator not being within the at least one range of positions, a graphical indicator is displayed in a graphical user interface that indicates that the endoscope is not properly oriented, which can guide the user to adjust the orientation of the endoscope. An example of this is illustrated in FIG. 6A. The indicator 620 is located outside of a range of positions indicated by target range indicator 624, and a notification 626 is provided that the endoscope is not properly aligned. The target range indicator 624 and a graphical object 628 located at the indicator 620 may help guide the user in adjusting the position of the endoscope to the correct position. As the user rotates the endoscope, the image processing system can track the position of the indicator 620 and update the position of the graphical object 628 according to the movement of the indicator 620.

At step 710, in accordance with the determined position of the rotational orientation indicator being within the at least one range of positions, the graphical user interface can be updated to display a graphical indicator that indicates that the endoscope is properly oriented. An example of such a graphical indicator is illustrated in FIG. 6D, in which the indicator 620 is located within the desired range of positions, as indicated by the indicator 620 being within the target range indicator 624, and a notification 630 is provided indicating that the scope is aligned.

In examples in which methods 500 and 700 are combined, determining suitability of an image for use in calibration at step 506 may include comparing the location of the endoscope orientation indicator with a predetermined range of positions according to step 706. A given image may be determined to be suitable if it meets the calibration marker requirements discussed above with respect to step 506 and the indicator position requirements of step 706 for a given stage of the calibration process. As such, calibration factors may be determined only from calibration images where the rotational orientation indicator is positioned in the desired range(s) of positions. As noted above, there can be multiple ranges of positions, and, as such, the calibration factors may be determined from sets of calibration images, each set being associated with a different range of endoscope rotational orientations. As explained above, the desired range(s) can be associated with endoscope rotational orientations typically used during surgery, and therefore, the calibration factors can be tailored more to the typical use that calibration factors generated from a calibration image captured without regard to the rotational orientation of the endoscope.

The graphical user interface (e.g., graphical user interface 600) can be used to provide the user information that can guide the user to capture images that are suitable for generating calibration parameters. Information can be provided to inform the user what to do next and/or to help the user rectify mistakes the user may be making that are preventing suitable images from being captured. Information for rectifying mistakes can be provided in any suitable manner, including with graphical indicators (such as target range indicator 624), textual indicators, pop-up messages, etc.

One example of information provided to the user for helping the user capture suitable images is described above with respect to target range indicator 624, which can indicate to the user that the endoscope is not oriented correctly. Another example is a notification to insert the endoscope into the calibration fixture to start capturing frames for calibration. This notification may be provided at the beginning of a calibration process prior to the image processing system having detected any calibration markers.

Another notification that may be provided is a notification that the endoscope is too close to the field of calibration markers. This notification may be provided when, for example, too few calibration markers are present in an image. An example of this is illustrated in FIG. 6B.

An image blurry notification may be provided when, for example, the image processing system is having trouble detecting calibration markers and/or when a blur-detection algorithm detects a sufficiently high degree of blurriness. An example of this is illustrated in FIG. 6C.

A notification to move the endoscope to a different port of the calibration fixture may be provided when a last region of the field of calibration markers has been successfully imaged from a first port of the calibration fixture. This notification may be provided in conjunction with range indicator 624 shifting to a different location if, for example, the second port is used for capturing images with the endoscope in a different rotational alignment.

Any of the above notifications can be based on analysis of each image received in step 504 or some subset of the images received in step 504. Optionally, a given notification may be provided only if the issue persists for some threshold period, such as, for example, for more than 50% of the frames for the last 2 seconds.

In some instances, multiple issues, each with its own notification, may be present at the same time and a precedence order of notifications may be followed based on the criticality of the issue and its impact on the frame quality. For example, where the field of calibration markers has not yet been detected (indicating that the user has not yet inserted the endoscope into the calibration fixture) and no calibration markers are detected in received images, the notification for the user to insert the scope into the calibration fixture may be provided but not a notification that the endoscope should be moved closer or that the image is blurry.

As noted above, method 500 can be performed periodically to generate calibration factors that account for changes in the optical attributes of the endoscopic camera from one time to the next. For example, method 500 can be performed at the beginning of each imaging session in case some aspect of the endoscopic camera that affects image distortion has changed since the last time calibration factors were generated. However, in some instances, it may be unnecessary to generate new calibration factors since predetermined calibration factors may be sufficient. This could be the case, for example, where an endoscopic imager has not been altered from one imaging session to the next or where any alteration (e.g., changing of endoscopes) has had sufficiently minimal impacts on distortion that predetermined calibration factors still work to remove distortion. As such, method 500 may include a process to check whether predetermined calibration factors are sufficient for correcting distortion. If they are, a full calibration process need not be formed, which can save time for the user.

Returning to FIG. 5, method 500 may include optional step 512, in which at least some of the images that were determined to be suitable at step 506 are used to check whether predetermined calibration factors are sufficient for use in correcting distortion in the images. If the predetermined calibration factors are determined to be sufficient, then the calibration process may end, and the predetermined calibration factors may be used for correcting distortion during a subsequent imaging session. If the factors are determined to be insufficient, then method 500 can proceed to step 510, in which new calibration factors are generated. Alternatively, the calibration process may return to step 502 for gathering additional or alternative images and may proceed through step 510 to generate new calibration factors.

In some variations, steps 502 through 508 and 512 are performed in a pre-calibration process. If the predetermined calibration factors are determined to be sufficient, then the pre-calibration process may end, and the image processing system may enter an imaging mode in which the predetermined calibration factors are used to undistort images generated by the endoscopic imager. If the predetermined calibration factors are determined to be insufficient, then the image processing system may enter a recalibration mode for generating new calibration parameters. The recalibration mode may include performing step 510 of method 500. The recalibration mode may include guiding a user through a process for capturing new or additional images for use in step 510, which can include performing steps 502 through 508. Optionally, images determined to be suitable during the pre-calibration process can be used for step 510 and steps 508 through 508 can be performed for gathering additional images for use in step 510.

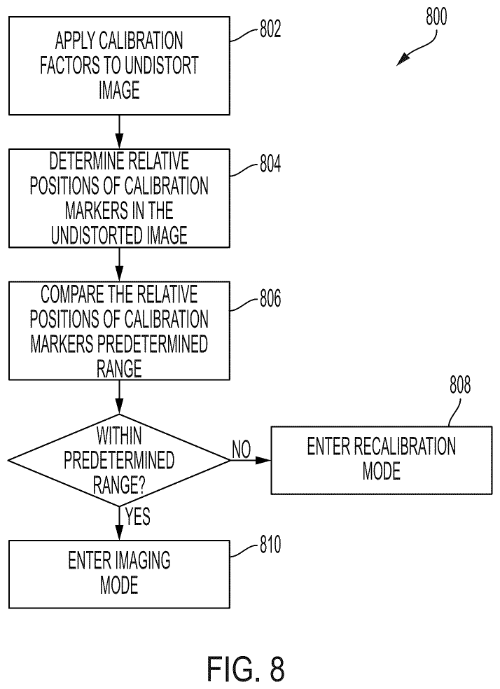

FIG. 8 is a flow diagram of an exemplary method 800 for determining whether predetermined calibration factors are sufficient for use in correcting distortion according to step 512 of method 500. Although method 800 is described below as being performed together with or as part of method 500, it should be understood that method 800 can be performed on its own or in combination with any other methods described here. Method 800 can be performed for a single image or may be performed for multiple images. Method 800 may be performed for at least one image of at least one region of a field of calibration markers. Method 800 may be performed for multiple images of each region of a field of calibration markers. Steps 502 through 508 of method 500 may be used to guide a user through steps for capturing images needed for performing method 800.

At step 802, predetermined calibration factors are used to undistort at least one image. The at least one image may be an image determined to be suitable for use in calibration for the endoscopic imager according to step 506 of method 500. The predetermined calibration factors may be calibration factors that were previously generated at step 510 of method 500, such as prior to a previous imaging session. However, method 800 is not limited to using calibration factors generated via method 500. Rather, the predetermined calibration factors may be made available to the image processing system performing method 800 (e.g., image processing system 116 of FIG. 1) in any suitable fashion, including by factory storing calibration factors on the image processing system, the camera control unit, or on the endoscopic camera, storing calibration factors in a database of calibration factors available to the image processing system (e.g., stored on the image processing system or stored on a server to which the image processing system is communicatively connected), or in any other suitable manner. In some variations, a user may select a camera model or other identifier from a list of available camera models or identifiers, and the calibration factors for the selected camera model/identifier may be used for step 802.

At step 804, relative positions of at least some of the calibration markers in the at least one undistorted image are determined. Step 804 may include detecting calibration markers, which can be done in similar fashion to the detection of the calibration markers in step 506 of method 500. Optionally, where method 800 is performed as part of method 500, the calibration markers detected in step 506 are used for method 800. Alternatively, calibration markers may be detected at two different times—once as part of step 506 for determining whether a given image is suitable and again in step 804 after the image distortion has been corrected with the predetermined calibration factors. Determining relative positions of calibration markers may include determining relative positions of one or more corresponding portions of the calibration markers. For example, the relative positions of corners of one or more calibration factors may be determined.

Optionally, determining the relative positions of calibration markers can include determining the degree to which calibration markers (or features thereof) that are known to be aligned are misaligned in the image. This may be done, for example, by determining a line that connects a pair of calibration markers (or features thereof) and determining how far other calibration markers (or features thereof) that are in line with the pair of calibration markers are from the line. For example, a line that connects a particular corner of a first calibration marker and a corresponding corner of a second calibration marker that is in line with the first calibration marker can be determined and the position of corresponding corners of other calibration markers between the first and second calibration markers can be compared to the line. Since the corners should line up with each other, the line should intersect all of the corners in an undistorted image. As such, determining a distance between corners and a line that should intersect the corners can be used to determine the sufficiency of calibration factors used to undistort the image in step 802. The line could be a horizontal line, and the distances between the line and the corners can be vertical distances. Alternatively, the line could be a vertical line, and the distances between the line and the corners can be horizontal distances. Optionally, multiple lines between multiple different calibration markers are used to determine alignment of calibration markers. For example, a horizontal line between corners of a first set of calibration markers may be used for a row of calibration markers, and a vertical line between corners of a second set of calibration markers may be used for a column of calibration markers.

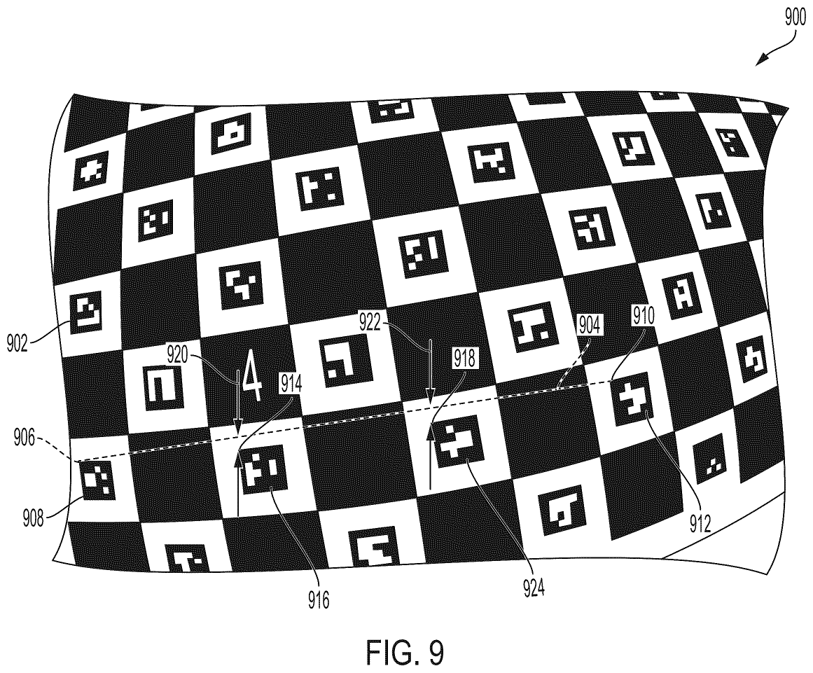

An example of determining the alignment of corners of calibration markers is illustrated in FIG. 9, which shows a portion of an endoscopic image 900 capturing a portion of a field of calibration markers 902. A line 904 connects a corner 906 of a first calibration marker 908 and a corner 910 of a second calibration marker 912. In the example of FIG. 9, the line 904 may be compared to corners of other calibration markers disposed between calibration marker 908 and calibration marker 912, such as corner 914 of calibration marker 916 and corner 918 of calibration marker 924. As shown in the example, the line does not intersect corners 914 and 918. The distances 920 and 922 between the line 904 and the respective corners 914 and 918 may be determined. Optionally, the relative positions determined according to step 804 are the distances 920 and 922 between the line 904 and the respective corners 914 and 918.

Optionally, determining the distance between a given corner of a given calibration marker and a line connecting two other calibration markers can include determining world frame-based distances in which the actual distances in the image are estimated based on the known dimensions of the calibration markers. This can include determining a per-pixel scaling factor that relates a pixel to a world-based measurement (e.g., X millimeters per pixel) by dividing the known size of a calibration marker (e.g., either of the calibration markers to which the line is connected and/or any of the other calibration markers in the image) by the pixel size of the calibration marker in the image. The distance from a given corner of a given calibration marker to a line can then be determined in pixels, and this distance can be scaled by the scaling factor to generate a world-frame distance (e.g., in millimeters) from the corner to the line.

At step 806, the relative positions of the calibration markers determined in step 804 are compared to at least one predetermined range. The predetermined range may be associated with satisfactory degrees of distortion. If the relative positions (e.g., distances of corners to the line) are within the predetermined range (or some suitable subset of relative positions is within the predetermined range), then it may be determined that the calibration factors used to undistort the image in step 802 are sufficient. For example, with respect to FIG. 9, the distances 920 and 922 may be compared to a predetermined range to determine whether the image has been sufficiently undistorted. If the distances 920 and 922 (or either distance or an average of the distances) are within the predetermined range, then the determination may be made that the predetermined calibration factors are sufficient. Conversely, if the relative positions do not satisfy the predetermined range, then the determination may be made that the predetermined calibration factors are insufficient.

At step 808, in accordance with the relative positions of the calibration markers (or some subset thereof) not being within the at least one predetermined range, the image processing system may enter a recalibration mode for generating new calibration parameters. Alternatively, if the relative positions of the calibration markers (or some threshold subset thereof) are within the at least one predetermined range, the image processing system may enter an imaging mode at step 810 in which the predetermined calibration factors are used to modify images captured by the endoscopic camera. Optionally, steps 804 and 806 are performed for a plurality of calibration images captured according to method 500, such as at least one image per region of a field of calibration markers. A determination of whether the predetermined calibration factors are sufficient for bypassing full calibration can be based on the distance measurements of all calibration images meeting the predetermined range. Alternatively, a predetermined percentage of the distance measurements (or some other threshold amount) meeting the predetermined range may be sufficient for deeming the calibration factors sufficient.

A graphical user interface, such as graphical user interface 600 of FIG. 6A, may provide one or more notifications to a user regarding the status of the pre-calibration process. For example, upon determining that the predetermined calibration factors are sufficient, a notification may be provided that the pre-calibration was successful. In contrast, should the predetermined calibration factors be determined to be insufficient, a notification may be provided that the pre-calibration failed and/or that the full calibration process is being performed.

According to an aspect, camera calibration can be performed without requiring calibration images that include calibration markers. For example, a machine learning model can be trained to estimate calibration factors based on images of a scene. An example of a suitable machine learning model is a Neural Radiance Fields (“NeRF”), such as described in Jeong et. al., Self-Calibrating Neural Radiance Fields, arXiv: 2018.13826v2 (2021), which is hereby incorporated by reference in its entirety.

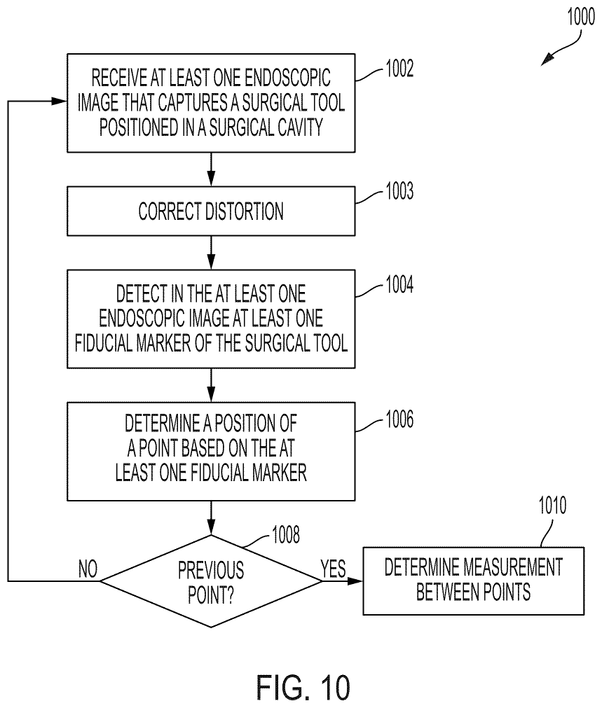

The calibration factors, whether predetermined or newly generated, are used by the image processing system for removing distortion from images generated during a surgical procedure on the patient. With the distortion removed, accurate measurements can be generated from the images. FIG. 10 illustrates an exemplary method 1000 for generating one or more measurements from images that have been corrected for distortion with calibration factors, such as calibration factors determined according to step 510 of method 500. Method 1000 can be performed by an image processing system, such as image processing system 116 of system 100. Method 1000 may be initiated by a user of the surgical tool, such by a user actuating a button 132 of pointer tool 122 of FIG. 1. Additionally or alternatively, method 1000 can be performed continuously or automatically.

At step 1002, at least one endoscopic image acquired by an endoscopic imaging system is received at the image processing system. For example, with reference to FIG. 1, image processing system 116 may receive at least one endoscopic image (which can be one or more single snapshot images or one or more frames of a video) from camera control unit 112. The endoscopic image captures a surgical tool positioned within a surgical cavity. For example, the endoscopic image may capture pointer tool 122 positioned within surgical cavity 104. At step 1003, the at least one endoscopic image is corrected to remove or reduce distortion using calibration factors, such as calibration factors determined according to step 510 of method 500.

At step 1004, at least one fiducial marker of the surgical tool is detected in the endoscopic image. For example, with reference to FIG. 1, the pointer tool 122 can include one or more fiducial markers 128. The image processing system may detect the fiducial marker by searching for and locating known visual patterns of the fiducial marker. In some variations, the fiducial marker is an ArUco marker, which includes an arrangement of light and dark blocks, the arrangement of which can uniquely identify a given fiducial marker relative to other fiducial markers of the surgical tool.

At step 1006, the image processing system determines a position of a point of interest of tissue based on determining a position of at least a portion of the surgical tool based on the detected fiducial marker. The image processing system may determine a position and/or orientation of the fiducial marker and may determine the position of the surgical tool (or a portion thereof) based on predefined relationships between the fiducial marker and the surgical tool. For example, with reference to FIG. 1, the image processing system may determine the location of a tip of the pointer tool 122 by accessing a database of predetermined positional relationships between the tip of the pointer tool 122 and the fiducial marker(s) 128. The position of the tip of the pointer tool 122 can be used as the position of the point of interest of tissue.

The fiducial marker 128 can be an ArUco marker, and the database of predetermined positional relationships (which can be stored in memory of the image processing system) can include a table of three-dimensional positions of the tip of the pointer tool 122 relative to corners of a laser marked perimeter of the ArUco marker. The image processing system may determine a location of at least one corner of the laser marked perimeter of the ArUco marker in two- or three-dimensional space and may use the three-dimensional positional relationship between the corner of the laser marked perimeter for the fiducial marker as listed in the table to calculate the position of the three-dimensional position of the tip of the pointer tool 122. The image processing system may do this for any of the fiducial markers that it is able to detect. The pointer tool 122 may include a plurality of fiducial markers that are each uniquely identifiable relative to the others (e.g., unique ArUco markers) and the table can include a set of three-dimensional positions associated with each of the plurality of fiducial markers 128.

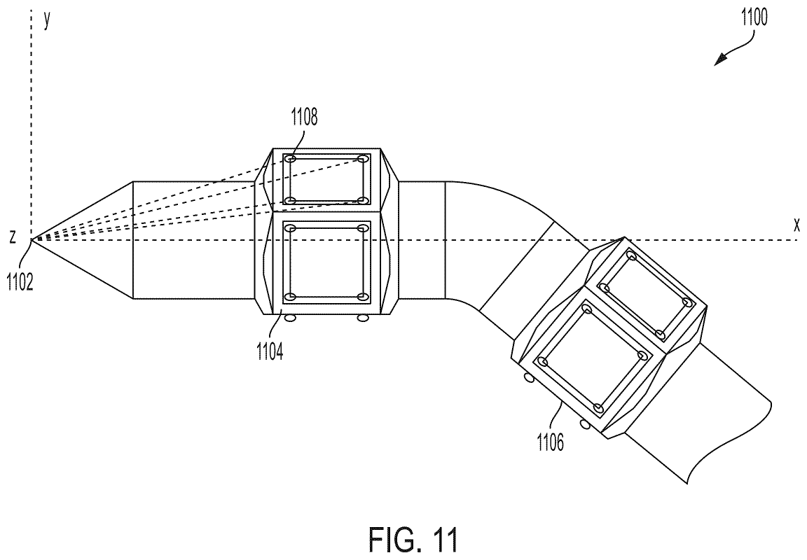

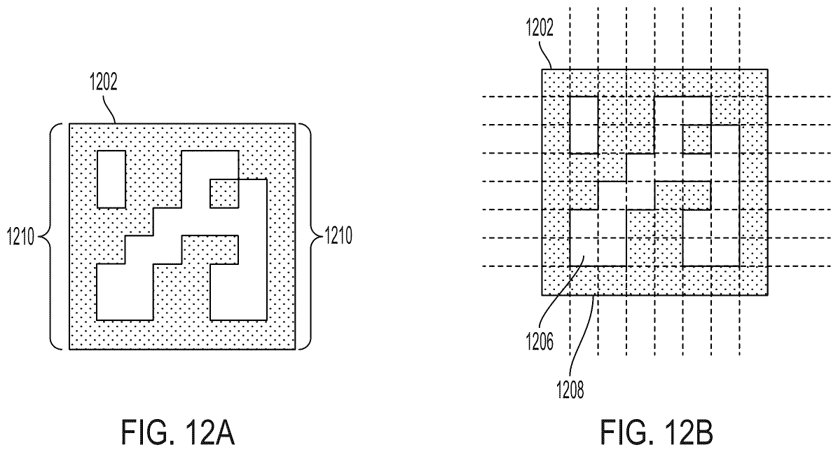

An example of the use of a fiducial marker to determine a position of a tip of a pointer tool, according to step 1006, is illustrated in FIGS. 11, 12A, and 12B. FIG. 11 illustrates an exemplary coordinate system for a pointer of an exemplary pointer tool. The exemplary pointer 1100 of FIG. 11 includes a tip 1102 and two sets of fiducial markers 1104 and 1106. Each set of fiducial markers 1104 and 1106 includes a plurality of corners 1108. In the example shown in FIG. 11, a coordinate system has been superimposed on the pointer 1100 to illustrate how the positions of corners 1108 can be used to determine the position of the tip 1102 of the pointer 1100. The tip 1102 of the pointer 1100 can represent the origin (0,0,0) of the coordinate system, and the coordinates of each of the corners 1108 of the sets of fiducial markers 1104 and 1106 represent the distances along the x-axis, y-axis, and z-axis (the z-axis is out of the page) from the tip. These distances are predetermined and may be stored in a database that is accessible to an image processing system. The example of FIG. 11 illustrates that when a three-dimensional position of any of the corners 1108 is determined, the position of the tip 1102 can be determined using predetermined positional relationships between the corners 1108 and the tip 1102.

FIGS. 12A and 12B illustrate an exemplary ArUco marker that can be disposed on a pointer tool (and/or can be used for the calibration markers). In the example of FIG. 12, an ArUco marker 1202 can include a plurality of dark color (e.g., black) and light color (e.g., white) blocks 1206 in a specific arrangement that allows the ArUco marker 1202 to be uniquely identified. The blocks of the ArUco marker 1202 are arranged in a grid. FIG. 12B shows the ArUco marker 1202 with a grid superimposed on the ArUco marker 1202 to better illustrate the plurality of blocks. The ArUco marker 1202 can include, for example, 64 blocks that are arranged on an 8×8 matrix. The ArUco marker 1202 can include a border 1208 that frames the ArUco marker 1202. In one or more examples, the border 1208 can be disposed on the first row, the last row, the first column, and the last column of the ArUco marker 1202. In the example of an 8×8 matrix, the border 1208 can be arranged to leave an internal 11×6 matrix. Each block 1206 of the internal 6×6 matrix can either be a dark block or a light block. The examples of an 8×8 matrix and 6×6 internal matrix are meant as examples only and should not be seen as limiting to the disclosure. Thus, in one or more examples, a particular ArUco marker can be configured in a variety of dimensions and grid layouts without departing from the scope of the present disclosure.

In one or more examples, the light and dark blocks can be arranged on the 6×6 internal matrix to provide the ArUco marker 1202 with a unique arrangement that can be used to uniquely identify the ArUco marker 1202. An image processing system can determine the arrangement of the blocks 1206 of the ArUco marker 1202 and can obtain the positions of the corners 1210 of the ArUco marker 1202 (e.g., the corners of the 8×8 matrix). The image processing system can use the determined arrangement of the blocks 1206 to extract the identity of the ArUco marker 1202. The image processing system can then access a database that includes predetermined positions of the corners of ArUco markers relative to the tip of a pointer tool and extract the predetermined positions of the corners 1210 of the identified ArUco marker 1202 relative to the tip of the pointer tool. For example, the image processing system can access a database that includes an (x,y,z) entry corresponding to each corner of each ArUco marker and can obtain the (x,y,z) entries for the corners of a given ArUco marker based on the identity of the ArUco marker extracted from its unique arrangement of blocks. The image processing system can combine the positions of the corners 1210 of the ArUco marker 1202 with the predetermined positions of the corners 1210 of the identified ArUco marker 1202 relative to the tip of the pointer tool to determine the position of the tip of the pointer tool.

Optionally, one or more machine learning models can be used in combination with one or more steps of process 1000 of FIG. 10. For example, one or more machine learning models can detect a pointer tool or other surgical tool in an endoscopic image received at step 1002 at the detection of the pointer tool can be used to trigger and/or facilitate the detection of fiducial markers in step 1004. A machine learning model can be trained using a supervised training process in which images or videos of the pointer tool are annotated with the precise location of the tool in the image, so that the machine learning model (for instance a convolutional neural network (CNN)) can recognize the presence and location of a pointer tool in a given image. A machine learning model can be trained to analyze endoscopic video to detect the presence of a pointer tool in the video, determine a location of the pointer tool in the video, and track the pointer tool in the video over time. Such a machine learning model can be a convolutional long short-term memory (convolutional LSTM) model that utilizes both spatial and temporal features of endoscopic video and is trained in a weakly-supervised regime (e.g., where each label indicates a presence or absence of one or more pointer tools without providing their locations).

In some examples, an image processing system, such as image processing system 116 of system 100, uses a machine learning model to detect a particular use of a pointer tool that is indicative of a need to determine a position of a tip of the pointer tool and, in response to such a detection, to automatically initiate one or more steps of process 1000 of FIG. 10. The machine learning model can be a deep learning model trained to simultaneously perform pointer tool tracking, rough localization of the tip of the pointer tool, and recognition of an action performed using the pointer tool. The deep learning model can be an “instrument-verb-target” model trained to process video to detect an instrument (e.g., a pointer tool), performing an action (e.g., pointing), with respect to a target (e.g., bony tissue of a joint). For example, the “instrument-verb-target” model may detect, in video, a pointer tool moving toward bony tissue of a joint and then stopping (e.g., for some period of time indicative of a pointing action by a user) and may determine that this activity of the pointer tool is indicative that the pointer tool is pointing to the bony tissue.

In some examples, an “instrument-verb-target” machine learning model can continuously process incoming video to detect use of the pointer tool to point to tissue. For example, referring to process 1000 of FIG. 10, images received at step 1002 can be processed by an “instrument-verb-target” machine learning model to detect use of a pointer tool to point to tissue. The detection of such an action can then trigger performance of step 1004, which is described in detail above.

In some examples, the “instrument-verb-target” machine learning model can identify a region of the image containing the tip region of the pointer tool and the fiducial marker(s) and this information can be used in step 1004 to reduce the amount of image data that is processed to locate the fiducial marker(s) in step 1004, which can make locating the fiducial marker faster than processing an entire image. In other words, instead of step 1004 including the processing of an entire image to locate the fiducial marker(s), processing may be limited to the region(s) of the image identified by the “instrument-verb-target” machine learning model. Optionally, one or more image enhancement techniques may be applied to the region(s) of the image identified by the “instrument-verb-target” machine learning model to improve the identification of the fiducial marker(s) in step 1004, which may also reduce the amount of processing relative to a process that applies image enhancement techniques to the entire image.

Although the above refers to the “instrument-verb-target” machine learning model detecting the use of a pointer tool, this is merely exemplary, and it should be understood that the “instrument-verb-target” machine learning model can be trained to detect the use of any tool, including, for example, a cutter, drill, or any other surgical tool. Additionally, the detection of a suitable action need not lead to (or only to) step 1004. In some examples, a notification associated with the detection of the action can be provided to the user. For example, the detection of the use of a pointer tool can lead to a display, on a graphical user interface of a function guide that guides the user in using the pointer tool, for example, to define a measurement point. In some examples in which the tool detected is a cutter or drill and the target is tissue that should be avoided, an alert may be provided to the user alerting the user that the cutter or drill is too close to the tissue. An example of a suitable “instrument-verb-target” machine learning model is described in Nwoye et al., Rendezvous: Attention Mechanisms for the Recognition of Surgical Action Triplets in Endoscopic Videos, arXiv: 2019.03223v2 (Mar. 3, 2022), which is incorporated by references in its entirety. The machine learning model can be trained with video data in which frames of the video data are labeled with suitable “instrument-verb-target” labels. For example, frames of respective training videos that include a pointer tool that is being used to point to a bony structure of a joint can be labeled with “pointer tool-pointing-bony structure.” The machine learning model can then be trained with such training videos to detect the use of a pointer tool to point to bony structure.

Returning to FIG. 10, at step 1008, a determination is made whether a previous point of interest of tissue has been defined. If not, method 1000 may return to step 1002 for determining a second point of interest of tissue (such as, in response to a user request). If a previous point of interest has been determined, then at step 1010, a measurement between the points may be determined based on the difference in position between the points. The measurement may be used in any suitable way, including by displaying the measurement in a graphical user interface, such as in an overlay on an endoscopic video feed.

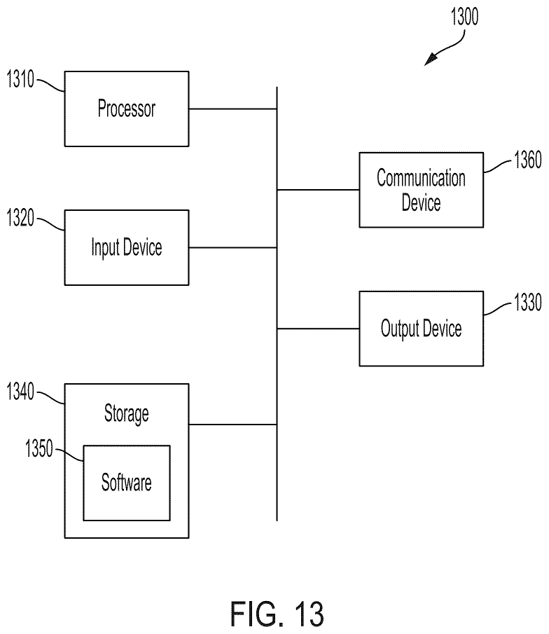

FIG. 13 illustrates an example of a computing system 1300 that can be used for one or more components of system 100 of FIG. 1, such as one or more of camera head 108, camera control unit 112, and image processing system 116. System 1300 can be a computer connected to a network, such as one or more networks of a hospital, including a local area network within a room of a medical facility and a network linking different portions of the medical facility. System 1300 can be a client or a server. System 1300 can be any suitable type of processor-based system, such as a personal computer, workstation, server, handheld computing device (portable electronic device) such as a phone or tablet, or dedicated device. System 1300 can include, for example, one or more of input device 1320, output device 1330, one or more processors 1310, storage 1340, and communication device 1360. Input device 1320 and output device 1330 can generally correspond to those described above and can either be connectable or integrated with the computer.

Input device 1320 can be any suitable device that provides input, such as a touch screen, keyboard or keypad, mouse, gesture recognition component of a virtual/augmented reality system, or voice-recognition device. Output device 1330 can be or include any suitable device that provides output, such as a display, touch screen, haptics device, virtual/augmented reality display, or speaker.

Storage 1340 can be any suitable device that provides storage, such as an electrical, magnetic, or optical memory including a RAM, cache, hard drive, removable storage disk, or other non-transitory computer readable medium. Communication device 1360 can include any suitable device capable of transmitting and receiving signals over a network, such as a network interface chip or device. The components of the computing system 1300 can be connected in any suitable manner, such as via a physical bus or wirelessly.

Processor(s) 1310 can be any suitable processor or combination of processors, including any of, or any combination of, a central processing unit (CPU), field programmable gate array (FPGA), and application-specific integrated circuit (ASIC). Software 1350, which can be stored in storage 1340 and executed by one or more processors 1310, can include, for example, the programming that embodies the functionality or portions of the functionality of the present disclosure (e.g., as embodied in the devices as described above), such as programming for performing one or more steps of method 500 of FIG. 5, method 700 of FIG. 7, method 800 of FIG. 8, and/or method 1000 of FIG. 10.

Software 1350 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1340, that can contain or store programming for use by or in connection with an instruction execution system, apparatus, or device.

Software 1350 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport computer-readable medium can include, but is not limited to, an electronic, magnetic, optical, electromagnetic, or infrared wired or wireless propagation medium.

System 1300 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

System 1300 can implement any operating system suitable for operating on the network. Software 1350 can be written in any suitable programming language, such as C, C++, Java, or Python. In various examples, application software embodying the functionality of the present disclosure can be deployed in different configurations, such as in a client/server arrangement or through a Web browser as a Web-based application or Web service, for example.

The foregoing description, for the purpose of explanation, has been described with reference to specific examples. However, the illustrative discussions above are not intended to be exhaustive or to limit the invention to the precise forms disclosed. Many modifications and variations are possible in view of the above teachings. The examples were chosen and described in order to best explain the principles of the techniques and their practical applications. Others skilled in the art are thereby enabled to best utilize the techniques and various examples with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application is hereby incorporated herein by reference.

1. A method for guiding a user through an endoscopic camera calibration process, the method comprising, at a computing system:
displaying visual guidance for guiding the user through the endoscopic camera calibration process, the visual guidance configured to guide the user to direct an endoscopic camera at a plurality of different regions of a field of calibration markers;
receiving images captured by the endoscopic camera while the user directs the endoscopic camera at the plurality of different regions of the field of calibration markers;
determining suitability of at least some of the images for use in calibration for the endoscopic camera;
in accordance with determining that at least one image of a respective region of the plurality of different regions of the field of calibration markers is suitable for use in calibration for the endoscopic camera, updating the visual guidance to indicate to the user that the respective region of the plurality of different regions of the field of calibration markers has been successfully imaged; and
determining calibration factors for calibrating the endoscopic camera based on images determined to be suitable for use in calibration for the endoscopic camera.

2. The method of claim 1, wherein the visual guidance comprises region indicators that correspond to the plurality of different regions of the field of calibration markers, and wherein updating the visual guidance to indicate to the user that the respective region of the plurality of different regions of the field of calibration markers has been successfully imaged comprises modifying a region indicator corresponding to the respective region of the plurality of different regions of the field of calibration markers.

3. The method of claim 1, wherein updating the visual guidance to indicate to the user that the respective region of the plurality of different regions of the field of calibration markers has been successfully imaged comprises displaying a graphical indicator within a field of view that comprises the respective region of the plurality of different regions of the field of calibration markers.

4. The method of claim 1, comprising, in accordance with determining that a given image of a given region of the plurality of different regions of the field of calibration markers is not suitable for use in calibration for the endoscopic camera, displaying at least one notification for guiding the user to adjust use of the endoscopic camera.

5. The method of claim 4, wherein the at least one notification comprises a notification that the endoscopic camera is too close to the field of calibration markers, that the endoscopic camera is out of focus, or that a rotation angle of the endoscopic camera is out of range.

6. The method of claim 1, wherein determining suitability of at least some of the images for use in calibration for the endoscopic camera comprises determining a rotation angle of an endoscope of the endoscopic camera relative to a camera head of the endoscopic camera and comparing the rotation angle to a predetermined range of suitable rotation angles.

7. The method of claim 1, wherein determining suitability of at least some of the images for use in calibration for the endoscopic camera comprises detecting calibration markers and comparing a number of detected calibration markers to a threshold number of calibration markers associated with suitability of an image for calibration.

8. The method of claim 1, comprising determining that each region of the plurality of different regions of the field of calibration markers has been successfully imaged, wherein the calibration factors are determined in response to determining that each region of the plurality of different regions of the field of calibration markers has been successfully imaged.

9. The method of claim 1, wherein the visual guidance is configured to guide the user to direct the endoscopic camera at the plurality of different regions of the field of calibration markers from a plurality of different endoscopic camera positions.

10. The method of claim 1, wherein the visual guidance indicates to the user an order in which to direct the endoscopic camera at the plurality of different regions of the field of calibration markers.

11. The method of claim 1, comprising:
undistorting at least one image of the received images based on predetermined calibration factors for the endoscopic camera;
determining relative positions of at least some of the calibration markers in the at least one undistorted image;
comparing the relative positions of the at least some of the calibration markers to at least one predetermined range;
in accordance with the relative positions of the at least some of the calibration markers not being within the at least one predetermined range, entering a recalibration mode that comprises the determination of the calibration factors for calibrating the endoscopic camera based on the images determined to be suitable for use in calibration for the endoscopic camera; and
in accordance with the relative positions of the at least some of the calibration markers being within the at least one predetermined range, entering an imaging mode in which the predetermined calibration factors are used to modify images captured by the endoscopic camera.

12. The method of claim 11, wherein the relative positions of the at least some of the calibration markers comprises relative positions of corresponding portions of the at least some of the calibration markers.

13. The method of claim 12, wherein determining the relative positions of at least some of the calibration markers in the at least one undistorted image comprises:
determining a location of a portion of a first calibration marker in the at least one undistorted image and a location of a corresponding portion of a second calibration marker in the at least one undistorted image; and
determining a distance from the location of the corresponding portion of the second calibration marker to a line extending from the location of the portion of the first calibration marker,
wherein the relative positions of at least some of the calibration markers in the at least one undistorted image comprises the distance from the location of the corresponding portion of the second calibration marker to the line extending from the location of the portion of the first calibration marker.

14. The method of claim 13, wherein the line is a horizontal line and the distance is a vertical distance or the line is a vertical line and the distance is a horizontal distance.

15. The method of claim 13, comprising determining a scaling factor based on a size of at least one of the first calibration marker and the second calibration marker in the at least one undistorted image and predetermined size information associated with the at least one of the first calibration marker and the second calibration marker, wherein the distance from the location of the corresponding portion of the second calibration marker to the line extending from the location of the portion of the first calibration marker is determined based on the scaling factor.

16. The method of claim 11, wherein the at least one image of at least a portion of the field of calibration markers comprises a plurality of images of the different regions of the field of calibration markers, wherein relative positions of calibration markers are determined and compared for each of the different regions of the field of calibration markers.

17. The method of claim 11, wherein the predetermined calibration factors comprise calibration factors determined during a previous calibration process.

18. The method of claim 11, wherein the predetermined calibration factors are associated with an endoscopic camera model or identifier.

19. A system comprising:
a calibration fixture; and
one or more processors and memory storing one or more programs for execution by the one or more processors, the one or more programs including instructions that, when executed by the one or more processors, cause the system to:
display visual guidance for guiding a user through a camera calibration process on a display, the visual guidance configured to guide the user to direct a camera at a plurality of different regions of the calibration fixture;
receive images captured by the camera while the user directs the camera at the plurality of different regions of the calibration fixture;
determine suitability of at least some of the images for use in calibration for the camera;
in accordance with determining that at least one image of a respective region of the calibration fixture is suitable for use in calibration for the camera, update the visual guidance to indicate to the user that the respective region of the calibration fixture has been successfully imaged; and
determine calibration factors for calibrating the camera based on images determined to be suitable for use in calibration for the camera.

20. A non-transitory computer readable storage medium storing instructions for execution by one or more processors of a computing system for causing the system to:
display visual guidance for guiding a user through a camera calibration process, the visual guidance configured to guide the user to direct a camera at a plurality of different regions of a calibration fixture;
receive images captured by the camera while the user directs the camera at the plurality of different regions of the calibration fixture;
determine suitability of at least some of the images for use in calibration for the camera;
in accordance with determining that at least one image of a respective region of the calibration fixture is suitable for use in calibration for the camera, update the visual guidance to indicate to the user that the respective region of the calibration fixture has been successfully imaged; and
determine calibration factors for calibrating the camera based on images determined to be suitable for use in calibration for the camera.
